SEQUENCE LISTING (27 sequences)

SEQ ID NO: 1 - DNA, 29 bp, Artificial Sequence
  PCR Primer for Sc-ACT1 5′
ccggaattcatggattctggtatgttcta

SEQ ID NO: 2 - DNA, 29 bp, Artificial Sequence
  PCR Primer for Sc-ACT1 3′
ccggaattcaagacagcacgaggagcgtc

SEQ ID NO: 3 - DNA, 30 bp, Artificial Sequence
  PCR Primer for Sc-GAP3 5′
atgatcagaattgctattaacggtttcggt

SEQ ID NO: 4 - DNA, 29 bp, Artificial Sequence
  PCR Primer for Sc-GAP3 3′
ttaagccttggcaacatattcgatcaagt

SEQ ID NO: 5 - DNA, 30 bp, Artificial Sequence
  PCR Primer for Sc-PMA1 5′
atgactgatacatcatcctcttcatcatcc

SEQ ID NO: 6 - DNA, 30 bp, Artificial Sequence
  PCR Primer for Sc-PMA1 3′
ttaggtttccttttcgtgttgagtagagac

SEQ ID NO: 7 - DNA, 30 bp, Artificial Sequence
  PCR Primer for Sc-TEF1 5′
atgggtaaagagaagtctcacattaacgtt

SEQ ID NO: 8 - DNA, 30 bp, Artificial Sequence
  PCR Primer for Sc-TEF1 3′
ttatttcttagcagccttttgagcagcctt

SEQ ID NO: 9 - DNA, 1300 bp, Candida maltosa
  Cm-ACT1 Promoter
gatctcggctgtgaatcgcaatttgccatgacacctctcgctatttccgaattacataagaccatcagtgaaagattggagagaaaaaaatgtgaacagaatagtcggagtttatattaaattcatcgtccaaaaaaacctgaatatcattggctctgcggtttattaaaaagtgtaccgccgacttatccgaacaattaaacgtaacatgactgcaaaaaaaaaccatgaagaaaacatttacaaaatatttgtaatatcgtcgaagataataaaccataccagacggaaagttggatgaaattgttgcaaaaataattatgctactcgttctcacgtttaaatatacacgtagatcaaaccagcaaacttctataaattgccatcgatccaccaaacatcgtcaaaaacaattttgtaactttattgcctctcatacgtttaaatttcaaaatcaataaatattaatcaaccgtgtaacaaaaaaaaaaaactgagatagtgcacagcccaaaaaggtttagtgattgcctccaaacatacataataggttatttttttcgttgaacgcatttcttgctcgctcaccgaatttctgccaacgaaacaaattttatatatttcatttttttctctttcatgtaattttttctttctttctgctttttcttttttttttcttttccttcccaatccccccctacattaatatattaattatcatttaaaatggacggtggtatgtttaaactatttcattgacttgattgatcgattgattgattggttgatttcttcaaagcacggactttttttctttctccattatttgatttttagattttgggtgatttttttgttttttttggggagtgactgatctaatctcaattcaggattatgggacgaagaagaatcgagagatggaatctagatatatcaatttcaatttttattgttttgatctcgagagtatttatggaaagatttgattgaacaactttttttttcattggcctggatcaattccgtccataaaagaaagagagctttacactgatcgattcattcatattttttacactggagtttcttcaaaaatcattggattctacccaatggcaatcctacatcttaaaaatcatcatttattacgagtttattggataatggtcactttaaattcttggtatcttgaatttgttttttttttttttttttttcagaaatttgtacccccttttaaaaatgttcagaatttttttttttttttcgtcaaacccacacacacacatttttc

SEQ ID NO: 10 - DNA, 3000 bp, Candida maltosa
  Cm-GAP3 Promoter
ctgcaggtgggatcttcagactgtttaatatttcttcaatatgatgggtcccataattagaaaccccaatactcttaacaatccccttggacacggcttcttccatgactttccaactttccaatcgtttttgtttcccaggtaatggtgaatggatcaataacaaatcgatatatttcaaatcaccaatttgatccaacatagtagtgatcgcatgtcttgtatttgttgtacccaattgactattccatagtttggtggtatagaaaaactctgaacgagggatatctggattatcgtgaaggaatttcgttatcccttctgccacttcttcttcgttgccgtacaaaactgcggtatcaaaatgtcgatatccgactttacaggcttcgtagacaatgctggccgttttatttcttggaatgtcgtaacagcctaaaccgattgaagggatgctatatccggaattgagtttgattaatcgaaatgacatgattgtgttgagtatattgaaagcaataattaatataaaaaaaagaggaacgaaaaaaaagagagatgttgaagtggttgggttatgtaagtacgtatttattcactgattattaattgctatcttaataatatttttttccctcccatttttactttttttggatatcttgtttgaaatgtgggggcaaaaaaaaaaaaaatttacatagccctattccataaaatataaatcttttatgtatatttgcaacatcgacacaatttgatatttccaaatactccaggtttttttttctttttcattcacagtctcgggattaagtgtgaaacccgggggaaatcgaaatttttttttttcagcattgtttatacacaatttcagtttgtccgaatacacccgcacgtgattcccccaaacaggcaaaaaaaaaaaaaaatgaatatatagtgagtacgtgtcccgcggctcaggaacctcttttttttttagaggtggtatgatgtgaagtattttttttttttcctttttccttttcctttttcattcacaccaccaccatatagaatttacttacgtcaggttatattctagacaacctttgtggtttttttttttaaagggaatttgagccactatgtccatagaaaactttttactgtaacgaaaatctatagtctgagataaaggggaaaatggtaaccacgtatttttttatttttttttggattcctataaccccgatatttatgttcggaattgtagatatatagatattccagattacttggctgtaatgtaggctatggaaatgatactactcatcaatataaacccattgacagtataagatagataattatactgtggtggtaccatataaaattaatatgttgatcaggtgcttttggcaacaccacgagctttgcgcaagttttttttttttgttcttttttgttttttgttggttgtttgatgcaaatggatgataatgccccgggcgcgggcgtgtgtgacgcaaatccaatagaaaaaattcacctggttaaacctattttcactgacaaatcaatttattttgccaaaagaaaaaaagaatatataataacccttgaatgtccaattggaattttttttctctttctaaaatttttctttctttctttcttttcttcttcttttcttctttacacaatcaattgactttaaacctcaattaaacaacacataactttcaaacttactttttaacatacaaaaaatggctattaaaattggtattaacggtttcggtagaatcggtagattagtcttgagaattgctttaggcagaaaagacattgaagttgttgccgtcaacgatccattcattgctgctgattacgctgcttacatgttcaaatacgattccacccacggtagatacaaaggtgaagtcaaatctgaaggtaacgatttagtcattgacggtaagaaaatccaagtcttccaagaaagagacccagctaacattccatggggtaaagaaggtgttgaatatgttattgactccactggtgttttcaccaagattgaaggtgctcaaaaacacattgatgctggtgccaaaaaagttatcatcactggttcatctgctgatgctccaatgttcgttgttggtgttaacgaagacaaatacaccccagacttgaaaatcatttctaacgcttcctgtaccactaactgtttagctccattagctaaagttatcaacgatactttcggaattgaagaaggtttgatgaccactgtccactccatcactgctacccaaaagactgttgacggtccttcccacaaagattggagaggtggtagaactgcttccggtaacattatcccatcttctactggtgctgctaaagccgtcggtaaagttatcccagaattaaacggtaaattgactggtatgtctttgagagttccaaccaccgatgtctccgttgttgacttgactgtcagattatctaaaccaaccacttacgaagaaatctctgaagctatcaagaaagctgctgatggtccattgaacggaatcttgggttacactgaagatgctgttgtctctactgacttcttgtcttctaactactcttctgttttcgatgctaaagctggtatcttgttgtccccaactttcgtcaaattgatctcttggtacgataacgaatacggttactctaccagagttgtcgacttattggaacacgttgccaaagtttccggttcctcttaactcagaaacaagttttagttgacattgtgtctgttttcttttattacataggttgttatatcaatatatgtttataaatacgtcttgaaaatcttgttttttttttttgtaaattttgtaaattttcatcttgtgcgggacaaaggacgagtggagaaaaaaaaaacgaaactttttttttcttttctccgaaattgtaaacaaaaacaacaacaacacctccatgtcggaa

SEQ ID NO: 11 - DNA, 3173 bp, Candida maltosa
  Cm-PMA1 Promoter
tctagaatttatattggtttctttctttttttttagatcgtttattaattaattagttaattaattacttcataacatgtaaattagatttaaccaaaaaaaagaaaagttaaagataatggctaagtagatgttaaagccaggttcaattgtttataatactcatcatcatcaatcaattaaattgcaaaaacaaacaaacccgttgaaaaaaggaaagaaaaaaaaaacacggctggtaataaatctttcatgtactggtcaattaattaaccgacgtaataagagatccttggataaatagtaagaatatccagcaatttacgtacgtaaatgaaacacaaatgaatgaatgctgaactttcatgacttaattgagtagtttagttggttggtatatgcgaaatttatttattccgataattattatcaataggttgtagcgggaaatttaaaaccaaacaggagattagaagcggcagaatgaaataaaaaaaaacatggccggcgaaaaaaaaaaggaaaaaaaggaaggaaaaaaaaacgaaaaagggtcgggtaaatctactcaacaaatattataataatgattgtttatttatctatggatgtttggatgaattaagtcaagtttgtgttatttcgtatgaaagagacatagttagagatagagatagatagacaaatagatttgagagatgaggtggttcagttacattacatttttttttcttaaaaactaaaaatatttcttatgtaactttccagtttattatattaataccaagaaagttatatgtaatatcagttgatattcaacaattgctgttacaattgtcaactctcaacttctaccttcccatttgaatatctctcttccagtcatatgagttgtattcaaaattttttttatttccgttcggcataattaattttgtgtcgtgggaatatgcacaatttataaaacaaaagcaaaatctaaattgagggaatttctgcagaagagtcaaaaaaaacataaagtcgtgtctcggaactcaaaaataacattttccatactaagattaaacgataacatttaaaaaaatccacaaatttgattggtcggaataaaaaataaaaatatccctcaccctaaagaaagaaaatttttttatttagttgagaaaaccgaataattttgtcctatgaggtaattaaatatttccattttgtgttattgtttattattttcctaaaccactttatcaaaaaaagaaaaagaaatttttcttcttttttggacaaaattaaaaattttttacaacctcttctaaaaaagaaaaacaacaacaacagaaaaacgacctccaaaaaaatcttacaaccaaaaatttaaatttttaattttccaaaggtaatataaaaaggataataaattcccttgattagatttttttttttaacgaattctttattcatttttcctttttccctttttttttttttcctttttttagatagtcaatcgaagttttacttttattaactttttttcctacccactaattcttactttcttttttttttcattcaaaaattttttaatagtattttaaaaaatataccatctcacacccccaaaaaagaaaaataaaaaggaattcatttttaataccctaattttttaatattagaattatagagagagaaaaagagacagaaaacaaaaacttatcatgagtgctactgatcctactaatgaaaagatcaataaagacatctccgatgatgaagatgaagatattgatcaattggttttagatttacaatctaatccaggtgctttagatgacgaagaagaagaagaagatcaagcttcttttaaagccgtccctgaagaattattacaaactgacccaagaactggtttatctgatgatgaagttcaaaaaagaagaaaaaagtatggtttgaatcaaatggctgaagaacaagaaaatttagtcatgaaattcgtcatgtttttcgttggtccaattcaattcgttatggaagccgctgctgttttagctgctggtttggaagattgggtcgattttggtgttatctgtgctttattggtattgaatgcttttgttggtttcatccaagaataccaagctggttctattgtcgatgaattaaaaaagactttagctaacgttgctttagttgttagaaatggtcaattggttgaaattccagccaatgaagttgttccaggtgatatcttgcaattggaagatggtaccgttatcccaactgatggtagaattgtttctgaagattgtttattacaagttgatcaatctgctattactggtgaatgtttagctgttgacaaaagatctggtgactcttgttactcttcttccactgttaaaactggtgaagcttttatggttgttaccgctactggtgacaacacttttgttggtagagctgctgctttagtcaacaaagcttccgctggtactggtcatttcactgaagttttgaacggtattggtactacattgttggttttcgtcattgttactttgttggttgtctgggttgcttgtttctacagaactgttagaattgttccaatcttgagatacactttggctatcaccattattggtgttccagtcggtttaccagctgtcgttaccactaccatggctgtcggtgctgcttacttggctaaaaaacaagctattgtccaaaaattgtctgctattgaatctttggctggtgtcgaaattttatgttctgataaaactggtactttgaccaagaataaattgtctttacatgaaccatacactgttgaaggtgttgaaccagatgacttgatgttgactgcttgtttggctgcttccagaaagaagaagggtttggatgctattgataaagctttcttgaaatctttgattaactacccaagagctaaagctgctttaccaaaatacaaagttattgaattccaaccttttgatcctgtctccaaaaaagttactgccattgttgaatcaccagaaggtgaaagaattatttgtgttaagggtgctccattattcgtcttgaagactgttgaagatgcccatccaatcccagaagatatccatgaaaactatcaaaacactgttgccgaatttgcttctaga

SEQ ID NO: 12 - DNA, 1675 bp, Candida maltosa
  Cm-TEF1 Promoter
ctgcagcagcttctactgctgccgctccaacattaggtgctgaatatactagcggtactggtaaattagttggtgtggttacattgactgatattttgggattatttgccacatcaaaaggtagaagaactgatccacaagctgcaagaaaccaaagaagaagaagttccacttccactacgagatcatctgttgatagtgcattaaacgctgaaggtgtgattaatccttctgccaccaccaccaccgatgccattcctggtaataacaattctagtcgtagagaaagtgttgatgcttcaagtgatgttttcagaaaatcatttactaaaccccaagaaaatgtattttccaaagagtaagggttccttctttcataacaaaaaaagaaaaacaatcaccgatttatttatttatttgcaatgctatttataatatattttgtagataaaaacaaatgaaaaatcttgttagctatgtatactactacatatatactacaataaaaacacaccaaaatgaaacgtgttttgcacaatttcgcacgactcagaggcatcgcatttctgtcctttttgtacgtcattgtaattttttttatgttattttttttacagcaagcaatccaaaaaaacaaaaaaaaaatgagagagaaaaaaatgagggggttgatttaaaaagatggtcaaaaatattcgtgacatattacataatcgatgagtttgatatggaacgaatattgatggttttggtctgaattgatatggtgtaagtatttgttggtgataattttatcaacataaactcaattccgctcaattgtacaaaattgaccttctttcgccttttgttcaatgccattttttccaataattttttttttcaaattttgccatccagcacaaagaaaaaaaaaatttacatgtccgacaactcaccggtgtttctgacaacaattgacaacaccagtctgtagacccaattggtaagtcaatgataactactacatctacctagttgttatcttttaacttaaaattagcaaagaaataataatggttatcattgaagatggtttcacaaaattaaacgaatacgtgtacgttttaccaaaaagatttttttttttctctttagtttttttttcgttgttcttcccatcactgaaaaatttttctccctctatataaatcaatcccatcaacgaaaattttttttcttcctttttgaattttttttttctcctttttttttctcctttttttttctccttttctttcttcatctaacttatatttaatcaatcatgggtaaagaaaaaactcacgttaacgtcgttgttattggtcacgtcgattctggtaaatctactaccaccggtcacttgatctacaagtgtggtggtattgacaaaagaaccattgaaaaattcgaaaaagaagctgctgaattaggtaaaggttctttcaaatacgcttgggtcttggataaattgaaggctgaaagagaaagaggtatcaccattgatatcgctttgtggaaattcgaaactccaaaataccacgttaccgttattgatgctccaggtcacagagatttcatcaaaaatatgattactggtacttctcaagctgattgtgctattttgattattgctggtggtactggtgaattc

SEQ ID NO: 13 - DNA, 1794 bp, Artificial Sequence
  Polyhydroxyalkanoate Synthetase Gene, ORF2S
atgtctcaaccatcttatggtccattgttcgaagctttggctcattacaatgataaattgttggctatggctaaagctcaaaccgaaagaactgctcaagccttgttgcaaactaacttggatgatttgggtcaagttttggaacaaggttctcaacaaccatggcaattgattcaagctcaaatgaattggtggcaagatcaattaaaattgatgcaacacactttgttaaaatctgctggtcaaccatctgaaccagttattactccagaaagatctgatagaagatttaaagctgaagcttggtctgaacaaccaatttatgattacttaaaacaatcctatttgttaactgctagacatttgttggcttctgttgatgctttggaaggtgtcccacaaaaatctagagaaagattgagattctttactagacaatacgtcaacgctatggctccatctaatttcttggctactaacccagaattgttaaaattgactttggaatccgatggtcaaaatttggttagaggtttggctttattggctgaagatttggaaagatctgctgatcaattaaacattagattgactgatgaatccgcttttgaattaggtagagatttggctttgactccaggtagagttgttcaaagaactgaattatatgaattaattcaatactctccaactactgaaaccgttggtaaaaccccagttttgatcgttccaccattcattaataaatattacattatggatatgagaccacaaaactccttggtcgcttggttggtcgctcaaggtcaaaccgttttcatgatttcctggagaaacccaggtgttgctcaagctcaaattgatttagatgattatgttgttgatggtgtcattgctgctttggatggtgttgaagccgctactggtgaaagagaagttcacggtattggttactgtattggtggtaccgctttgtctttagctatgggttggttggccgccagaagacaaaaacaaagagttagaactgctactttgtttactactttgttggatttctcccaaccaggtgaattgggtatttttattcatgaaccaattatcgccgccttagaagcccaaaatgaagctaaaggtattatggatggtagacaattggccgtctccttctctttgttgagagaaaactctttatattggaattactatattgattcttacttaaaaggtcaatctccagttgcttttgatttgttgcactggaactctgattctactaatgttgccggtaaaactcataactctttgttgagaagattatatttggaaaatcaattggttaaaggtgaattaaaaattagaaacactagaattgatttaggtaaagttaaaactccagttttgttggtttctgccgttgatgatcacattgctttatggcaaggtacctggcaaggtatgaaattgttcggtggtgaacaaagatttttattggccgaatccggtcatattgctggtattattaatccaccagctgctaacaaatacggtttctggcacaatggtgctgaagctgaatctccagaatcttggttggctggtgccacccatcaaggtggttcctggtggccagaaatgatgggttttattcaaaacagagatgaaggttctgaaccagtcccagccagagtcccagaagaaggtttggctccagctccaggtcactatgtcaaagttagattaaacccagttttcgcttgtccaaccgaagaagatgctgcttctaaattgtaa

SEQ ID NO: 14 - DNA, 218 bp, Candida maltosa
  terminator ALK1t
atagatggatttttcttttttatgtgtatttccggttaataaatgtttaaattttttttttaataaaaatatttgtagttatttatatgcaaaaaaaaaaaatattcaaagcaatcttcctttctttctttatctttcccccatgctaaggtctaaaacaccacaacttaaaacccaacttaaccgtataatactaagatcaatctccaaagatgcat

SEQ ID NO: 15 - DNA, 1017 bp, Candida maltosa
  promoter ALK1p
atgcatgaacaggatttaatcccaagaaaaaagtctattttctattttcacaaggaaactggaaaaacctttttgtgttttgaagtagctccgtaataacctgtaaaaaaataaattttgaagatttgacttgctgatgaaaatgctatcagtgtagctctagacttgatactagactatgatggcaacacatggtggtcaacgtgcaagacatcacccaatgagaagactgctaaccagaaaaaaaaggggacaaaagaaaaactcgagagaaaaagtcaaattggtgtaaaattggctatttttggtactttcctaatggggaaattaattgtttaaaattccagtttttccagagttaagatttcgaccaattatttttaatccatatgatcttcatcattatcaacttgtgaaaaataataatcgaggtacgtttaatacgagatattagtctacggctatgaatgttggatatacttcattgacgatcagaagcttgattggttattcaggtgcatgtgtggatataaacccaacaaattatctagcaactgtgccttccccacattggtcaaagaaaccctaaagcaaattaaaatctggataaataaatcattcatttcacattttccggttagtataaggttttttaaatttttttttacagtttagccctttcaattaccaaatacggtaacaatgtgctttgtaacatgcaggggattttctccgttgctgttttctccacatgcttttaatgtgtaataaattaaaaaaattacaaagaaaaaccggcatataagcatcggagtttacattgttaactaactgcaaaatggcgatgtttcaaatcaacaaaatttaaaaaaaccccaaaaaaaaagtatcatataaattaaactcaaaatccttttgattgcataaaatttttaaatctcttcttttttttcttttttactttcttatctattctattctttttttatatatctaattcatttataacatctggtc

SEQ ID NO: 16 - DNA, 46 bp, Artificial Sequence
  PCR Primer for ALK1p 5′
tttttcagctggagctcgtcgacatgcatgaacaggatttaatccc

SEQ ID NO: 17 - DNA, 39 bp, Artificial Sequence
  PCR Primer for ALK1p 3′
ccggaattccatatgcagatgttataaatgaattagata

SEQ ID NO: 18 - DNA, 32 bp, Artificial Sequence
  PCR Primer for ALK1t 5′
cggaagcttatagatggatttttcttttttat

SEQ ID NO: 19 - DNA, 46 bp, Artificial Sequence
  PCR Primer for ALK1t 3′
tttttgatatcgagctcgtcgacatgcatctttggagattgatctt

SEQ ID NO: 20 - DNA, 47 bp, Artificial Sequence
  PCR Primer for Cm-ACT1p 5′
cgcggatccgaattcgtcgacatgcatggatctcggctgtgaatcgc

SEQ ID NO: 21 - DNA, 35 bp, Artificial Sequence
  PCR Primer for Cm-ACT1p 3′
gcgggatcccatatgtatccaataaactcgtaata

SEQ ID NO: 22 - DNA, 35 bp, Artificial Sequence
  PCR Primer for Cm-GAP3p 5′
atggctattaaaattggtattaacggtttcggtag

SEQ ID NO: 23 - DNA, 35 bp, Artificial Sequence
  PCR Primer for Cm-GAP3p 3′
agaagcattggagataatcttcaagtctggagtgt

SEQ ID NO: 24 - DNA, 34 bp, Artificial Sequence
  PCR Primer for Cm-PMA1p 5′
gaatatctctcttccagtcactcgagttgtattc

SEQ ID NO: 25 - DNA, 28 bp, Artificial Sequence
  PCR Primer for Cm-PMA1p 3′
ctcatatgaagtttttgttttctgtctc

SEQ ID NO: 26 - DNA, 34 bp, Artificial Sequence
  PCR Primer for Cm-TEF1p 5′
gcgggatcctcgagtaagggttccttctttcata

SEQ ID NO: 27 - DNA, 38 bp, Artificial Sequence
  PCR Primer for Cm-TEF1p 3′
ttttctttacccatatgtgattaaatataagttagatg

(19) United States
(12) Patent Application Publication
(10) Pub. No.: US 2008/0044878 A1
(43) Pub. Date: Feb. 21, 2008

(54) Novel Promoters

(76) Inventors: Tetsuya Nagaoka, Hyogo (JP); Satoru Yokomizo, Hyogo (JP); Kenji Miyamoto, Kanagawa (JP); Fumio Osakada, Okayama (JP); Keiji Matsumoto, Hyogo (JP); Masamichi Takagi, Tokyo (JP); Akinori Ota, Saitama (JP)

Correspondence Address: KENYON & KENYON LLP, 1500 K STREET N.W., SUITE 700, WASHINGTON, DC, 20005, US

(21) Appl. No.: 10/505,229

(22) PCT Filed: Apr. 8, 2003
(86) PCT No.: PCT/JP03/04426

§ 371 Date: Apr. 20, 2005

(30) Foreign Application Priority Data
Apr. 8, 2002 (JP) 2002-105240

(51) Int. Cl. C07H 21/04; C12N 1/00; C12P 1/02

(52) U.S. Cl. 435/171; 435/255.4; 536/23.1; 536/24.1; 536/24.2

(57) ABSTRACT

The present invention provides a production method of a copolymeric polyester which comprises culturing an ACT1 gene promoter shown under SEQ ID NO: 9, a GAP3 gene promoter shown under SEQ ID NO: 10; a PMA1 gene promoter shown under SEQ ID NO: 11, and, a TEF1 gene promoter shown under SEQ ID NO: 12; a plasmid which contains the gene expression unit comprising said promoter; a transformed cell as resulting from transformation of the said plasmid; and said transformed cell.

### TECHNICAL FIELD

The present invention relates to a novel promoter enabling gene expression in yeasts belonging to the genus Candida. More particularly, it relates to a promoter enabling constitutive and highly efficient gene expression in yeast belonging to the genus Candida without dependence on the induction conditions such as culture conditions and medium conditions.

### BACKGROUND ART

Advances in gene recombination technology have made it possible to produce useful proteins and useful chemicals, among others, using microorganisms. Gene Recombination systems in which Escherichia coli and Bacillus subtilis, which are prokaryotes, have been positively developed, and various useful substances have been produced using host-vector systems involving Escherichia coli, in particular. However, there are problems: proteins produced in Escherichia coli may form insoluble granules in cells and they cannot be undergone glycosylation, which is a characteristic feature of eukaryotes.

The development of systems in which yeasts, which are eukaryotes, are used as hosts has also advanced. Yeasts have been long utilized in brewing and baking and once have been produced for use in feedstuffs and, thus, their high safety has been guaranteed. Their ability to glycosylate proteins produced therein is one of the features distinguishing them from prokaryotes.

Host-vector systems have been developed not only in Saccharomyces cerevisiae, on which genetic findings are abundant, but also in the genera Schwanniomyces, Kluyveromyces, Pichia, Hansenula, Yarrowia and Candida (Klaus Wolf: Nonconventional Yeasts in Biotechnology, published by Springer).

Among those yeasts, some can grow utilizing straight hydrocarbon chains (n-alkanes) as the only carbon source. They are Candida maltosa, Candida tropicalis and the like, which belong to the genus Candida, Hansenula polymorpha, and Yarrowia lipolytica, among others. These yeasts have an enzyme system for oxidizing n-alkanes at the terminus thereof and thus can utilize them as energy sources by decomposing the long-chain carboxylic acids resulting from such oxidation to give acetyl-CoA, which serves as a substrate of the TCA cycle, by means of the peroxisome β oxidation system.

Such n-alkane-utilizing yeasts not only can grow on straight hydrocarbons as carbon sources but also are resistant to hydrophobic substances which inhibit the growth of general yeasts. Therefore, they are promising hosts for providing the field of reaction for producing useful substances by conversion of hydrophobic chemical substances. Thus, by constructing a gene expression system in such yeast, it becomes possible to construct a novel useful chemical substance production system in which no hazardous byproducts are produced and no energy is wasted.

Positive efforts have been made to construct gene expression systems in Candida maltosa among such n-alkane-utilizing yeasts. M. Kawamura et al. discovered a region causing high-efficiency transformation (transformation ability; hereinafter referred to as “TRA” for short) in Candida maltosa (M. Kawamura, et al., Gene, vol. 24, 157 (1983)). It was revealed that this region includes a sequence involved in autonomous replication in Candida maltosa (autonomously replicating sequence; hereinafter referred to as “ARS” for short) and a centromere sequence (hereinafter referred to as “CEN” for short). Until now, a low-copy-number vector having the whole of the TRA region and a high-copy-number vector deprived of the CEN region and expected to allow high-level expression of transgenes have been developed (M. Ohkuma, et al., Mol. Gen. Genet., vol. 249, 447 (1995)).

A promoter capable of functioning in Candida maltosa is required for gene expression in that yeast, and a plurality of promoters are now available therefor. Candida maltosa produces, in the presence of alkanes, high levels of enzymes involved in the n-alkane oxidation system. In particular, transcription of the genes encoding cytochrome P450 which can be participates in early-stage oxidation of alkanes (hereinafter such genes being referred to as “ALK” for short) are strongly induced (M. Ohokuma, et al., DNA and Cell Biology, vol. 14, 163 (1995)) and the transcription of genes encoding the β oxidation system are also induced (Y. Masuda, et al., Gene, vol. 167, 157 (1995)). The promoter of the ALK1 gene among the ALK gene group is most strongly induced by n-alkanes and can be utilized in gene expression. The promoter of the ALK2 or ALK5 gene is suited for use in the presence of fatty acids.

The promoter of the gene for phosphoglycerate kinase (hereinafter referred to as “PGK” for short) known as an enzyme involved in the glycolytic pathway is known to induce potent gene expression in the presence of glucose. The PGK promoter of Candida maltosa has been cloned by Y. Masuda et al. (Y. Masuda, et al., Curr. Genet., vol. 25, 412 (1994)). Furthermore, the GAL promoter having potent gene expression inducing activity in the presence of galactose has also been cloned (S. M. Park, et al., Yeast, vol. 13, 21 (1997)). The ALK promoter, PGK promoter and GAL promoter cloned as mentioned above can be utilized in gene expression in Candida maltosa.

In cases where such a carbon source as glucose is used, however, the ALK promoter will hardly function. When fatty acids or n-alkanes are used as carbon sources, the PGK promoter will hardly function. Therefore, carbon sources suited for the production of a useful substance in Candida maltosa are not always adequate for potent gene expression in the production of that substance. Furthermore, the GAL promoter is induced only when galactose is used as the carbon source; hence, the GAL promoter may be said to be unsuited for commercial production since the use of galactose, which is expensive, is required.

Thus, a novel promoter capable of potent gene expression without any restriction as to the kind of carbon source has been desired for high-efficiency production of useful substances in Candida maltosa.

As regards Saccharomyces cerevisiae, on the other hand, promoter of the alcohol dehydrogenase 1 gene, glyceraldehyde-3-phosphate dehydrogenase 3 gene (hereinafter referred to as “GAP3” for short), PGK and GAL, among others, have already been cloned and have been widely utilized as promoters for the expression of foreign genes. It has already been revealed that the activity of these promoters is powerful when that yeast is used as the host. Thus, high expression levels can be expected even when Candida maltosa is used as the host. However, such promoter region genes have not been cloned as yet in Candida maltosa; therefore, the cloning thereof has been awaited.

Unlike Saccharomyces cerevisiae, however, no sexual generation is known of yeasts of the genus Candida. In many of them, the genome is diploid. Furthermore, it was reported that there is an abnormality in the reading of the genetic code. It is reported that the CUG codon generally encoding leucine is read as the codon for serine in Candida cylindraceae (Y. Kawaguchi, et al., Nature, vol. 341, 164 (1989)) or in Candida maltosa (H. Sugiyama, et al., Yeast, vol. 11, 43 (1995)). In this manner, yeasts of the genus Candida may be said to have properties markedly different from those of Saccharomyces cerevisiae and, therefore, it has remained unknown whether the Saccharomyces cerevisiae-derived promoter region as such can show comparative promoter activities in yeasts of the genus Candida as well.

### SUMMARY OF THE INVENTION

In view of the above-mentioned state of the art, it is an object of the present invention to construct a promoter enabling potent gene expression in yeast of the genus Candida, especially in Candida maltosa without being influenced by the kind of carbon source and thus provide a novel promoter enabling constitutive and high-efficiency production of useful substances.

As a result of intensive investigations made by them, the present inventors succeeded for the first time in identifying the actin synthetase 1 gene (hereinafter referred to as “ACT1” for short) promoter, glyceraldehydes-3-phosphate dehydrogenase 3 gene (hereinafter referred to as “GAP3” for short) promoter, protoplasmic membrane proton ATPase 1 gene (hereinafter referred to as “PMA1” for short) promoter and translational elongation factor 1 gene (hereinafter referred to as “TEF1” for short) promoter of Candida maltosa, and found that those promoters are at comparable or superior in promoter activity to the existing ALK promoter, PGK promoter and GAL promoter. Such findings have now led to completion of the present invention.

Thus, the present invention relates to an ACT1 gene promoter

which comprises a DNA selected from among the following

(a) to (d):
(a) a DNA shown under SEQ ID NO: 9;
(b) a DNA containing the base sequence shown under SEQ ID NO: 9 and having promoter activity;
(c) a DNA containing a base sequence derived from the base sequence shown under SEQ ID NO: 9 by deletion, substitution or addition of at least one base and having promoter activity;
(d) a DNA derived from a yeast belonging to genus Candida, which hybridizes with base sequence of SEQ ID NO: 9 under stringent condition and has a promoter activity.

In addition, the present invention relates to a GAP3 gene promoter

which comprises a DNA selected from among the following

(a) to (d):
(a) a DNA shown under SEQ ID NO: 10;
(b) a DNA containing the base sequence shown under SEQ ID NO: 10 and having promoter activity;
(c) a DNA containing a base sequence derived from the base sequence shown under SEQ ID NO: 10 by deletion, substitution or addition of at least one base and having promoter activity.
(d) a DNA derived from a yeast belonging to genus Candida, which hybridizes with base sequence of SEQ ID NO: 10 under stringent condition and has a promoter activity.

Furthermore, the present invention relates to a PMA1 gene promoter

which comprises a DNA selected from among the following

(a) to (d):
(a) a DNA shown under SEQ ID NO: 11;
(b) a DNA containing the base sequence shown under SEQ ID NO: 11 and having promoter activity;
(c) a DNA containing a base sequence derived from the base sequence shown under SEQ ID NO: 11 by deletion, substitution or addition of at least one base and having promoter activity.
(d) a DNA derived from a yeast belonging to genus Candida, which hybridizes with base sequence of SEQ ID NO: 11 under stringent condition and has a promoter activity.

Moreover, the present invention relates to a TEF1 gene promoter

which comprises a DNA selected from among the following

(a) to (d):
(a) a DNA shown under SEQ ID NO: 12;
(b) a DNA containing the base sequence shown under SEQ ID NO: 12 and having promoter activity;
(c) a DNA containing a base sequence derived from the base sequence shown under SEQ ID NO: 12 by deletion, substitution or addition of at least one base and having promoter activity.
(d) a DNA derived from a yeast belonging to genus Candida, which hybridizes with base sequence of SEQ ID NO: 12 under stringent condition and has a promoter activity.

Furthermore, the present invention relates to a DNA which comprises any one of the promoter mentioned above and a structural gene joined to the promoter sequence downstream therefrom.

Furthermore, the present invention relates to a gene expression unit

which comprises the DNA mentioned above and a terminator.

Furthermore, the present invention relates to a plasmid which contains the gene expression unit mentioned above.

Furthermore, the present invention relates to a transformed cell as resulting from transformation of the DNA or the plasmid mentioned above into a host cell.

Furthermore, the present invention relates to a method of producing the copolymeric polyester resulting from copolymerization of 3-hydroxybutyric acid and 3-hydroxyhexanoic acid

which comprises culturing the transformed cell wherein the structural gene is an Aeromonas caviae-derived gene encoding an enzyme involved in the synthesis of the copolymeric polyester resulting from copolymerization of 3-hydroxybutyric acid and 3-hydroxyhexanoic acid.

### DETAILED DESCRIPTION OF THE INVENTION

The ACT1 gene promoter, GAP3 gene promoter, PMA1 gene promoter and TEF1 gene promoter of Candida maltosa are novel promoters the sequences of which have been identified for the first time by the present inventors. In the following, they are described in detail.

### (1) Cloning of Novel Promoters

The ACT1 gene encodes one of the enzymes involved in the synthesis of actin, which constitutes the cytoskeleton, the GAP3 gene for one of glycolytic pathway enzymes, the PMA1 gene for one of protoplasmic membrane proteins, which is said to account for about 10% of the protoplasmic membrane proteins in yeast of the genus Saccharomyces, and the TEF1 gene for one of translational elongation factors in protein synthesis, and all the corresponding promoters of Saccharomyces cerevisiae are known as potent promoters in that yeast.

For cloning the promoter regions of the ACT1 gene, GAP3 gene, PMA1 gene and TEF1 gene of Candida maltosa by hybridization, the present inventors first amplified, by PCR method, partial DNA sequences of the corresponding structural genes from Saccharomyces cerevisiae belonging to a relatively allied genus, and used them as probe fragments. For example, the sequence of the ACT1 gene of Saccharomyces cerevisiae has already been reported (Daniel, H. M. et al., Int. J. Syst. Evol. Microbiol., vol. 51, 1593 (2001)). Based on that sequence, PCR primers for amplifying the ACT1 gene of Saccharomyces cerevisiae can be synthesized. The sequence of the GAP3 structural gene of Saccharomyces cerevisiae has already been reported (Holland, M. J. et al., J. Biol. Chem., vol. 254, 5466 (1979)). Based on that sequence, PCR primers for amplifying the GAP3 gene of Saccharomyces cerevisiae can be synthesized. Further, the sequence of the PMA1 gene of Saccharomyces cerevisiae has already been reported by Capieaux, E. et al. (J. Biol. Chem., vol. 264, 7437 (1989)). Based on that sequence, PCR primers for amplifying the PMA1 structural gene of Saccharomyces cerevisiae can be synthesized. The sequence of the TEF1 structural gene of Saccharomyces cerevisiae has already been reported by Cottrelle, P., et al. (J. Biol. Chem., vol. 260, 3090 (1985)). Based on that sequence, PCR primers for amplifying the TEF1 structural gene of Saccharomyces cerevisiae can be synthesized.

The chromosomal DNAs of Saccharomyces cerevisiae and Candida maltosa can be isolated using commercial reagents, for instance. By using the thus-isolated chromosomal DNA of Saccharomyces cerevisiae as a template for PCR and carrying out PCR using the DNA primers synthesized as mentioned above, it is possible to amplify a part each of the ACT1, GAP3, PMA1 and TEF1 structural genes corresponding to each respective primer combination. Hybridization detection probes can be prepared by labeling the thus-amplified DNA fragments with a radioactive compound or alkaline phosphatase, for instance.

The lengths of fragments presumably containing the respective desired promoter sequences can be estimated by isolating the Candida maltosa chromosomal DNA, fragmenting it with an appropriate restriction enzyme to subject to agarose gel electrophoresis, and carrying out Southern hybridization using thus-obtained DNA with the above-mentioned detection probes. For each promoter, a gene library can be constructed by cleaving the Candida maltosa chromosome with the restriction enzyme used for the above Southern hybridization, and using a cloning vector. This library is transformed so that an appropriate number of colonies may appear on an antibiotic-containing agar medium and then cultured on a nitrocellulose membrane, and this membrane is subjected to alkaline denaturation, neutralization, washing and drying and then to hybridization, whereby a chromosomal DNA fragment containing each desired promoter of Candida maltosa can be cloned. Each DNA fragment obtained in this manner contains not only the respective promoter region but also the respective structural gene. Therefore, in view of their similarity (homology) to those corresponding known promoters or structural genes whose base sequences are known, such as Saccharomyces cerevisiae, the promoter regions can be identified and distinguished from the structural genes using general techniques to determine the base sequences of the promoters.

In the manner mentioned above, the novel ACT1 gene promoter, GAP3 gene promoter, PMA1 gene promoter and TEF1 gene promoter of the invention can be obtained. The base sequences of these promoters are shown under SEQ ID NOs: 9, 10, 11 and 12, respectively.

The above-mentioned promoters of the present invention may be obtained by using the techniques mentioned above. Since the base sequences thereof have been identified, it is also possible to obtained them by chemical synthesis.

The promoters of the present invention may be not only the above DNAs shown under SEQ ID numbers: 9, 10, 11 and 12 but also DNAs comprising a DNA containing any of the sequences mentioned above or a DNA derived from any of the sequences mentioned above by deletion, substitution or addition of at least one base. Furthermore, it may be a DNA derived from a yeast belonging to genus Candida, which hybridizes with the base sequence of above SEQ ID numbers under stringent condition and has a promoter activity.

The “DNA comprising a DNA containing any of the sequences mentioned above” or “DNA derived from any of the sequences mentioned above by deletion, substitution or addition of at least one base” means any DNA derived by the deletion, addition, insertion and/or substitution of a number of bases of the order which can be deleted, added, inserted and/or substituted by the methods well known in the art, for example as described in, inter alia, Protein, Nucleic Acid, Enzyme, Supplemental Issue: Gene Amplification PCR Technology TAKKAJ 35 (17), 2951-3178 (1990) or Henry A. Erlich (ed.), PCR Technology (the translation edited by Ikunoshin Kato) (1990).

The “DNA derived from a yeast belonging to genus Candida, which hybridizes with the base sequence of above SEQ ID numbers under stringent condition” means a DNA obtained by colony hybridization, plaque hybridization, Southern hybridization or the like hybridization technique.

The DNA of the present invention preferably comprises a base sequence showing a homology of not less than 80% to the base sequence shown under above SEQ ID number. The homology is preferably not less than 90%, more preferably not less than 95%, still more preferably not less than 98%.

The “homology” is calculated by aligning two nucleotide sequences to be compared in the optimum format, counting the matched base (A, T, C, G, U or I) positions between the two sequences, dividing the count by the total number of bases to be compared, and multiplying the product by 100. Specifically, this calculation can be made using an analytical software such as Hitachi Soft Engineering's DNASIS, Software Development's GENETYX, or Finland CSC's Clustal X, for instance.

These DNAs may be easily obtained by the deletion, addition, insertion and/or substitution, using the above well-known methods in the art, of at least one base to the DNA shown under above SEQ ID numbers, or by carrying out the hybridization according to the methods described in Molecular Cloning, 2nd Edition (Cold Spring Harbor Laboratory Press, 1989).

In the present invention, the promoter “having a promoter activity” means the promoter whose enzyme activity, measured by the function analysis mentioned hereinafter, is not less than 50% relative to that of the DNA of the above SEQ ID number measured under the same condition. The enzyme activity of the promoter is preferably not less than 70%, more preferably not less than 80%, particularly preferably not less than 90%.

### (2) Analysis of Promoter Functions

The functions of the novel promoters can be analyzed by quantitating the mRNAs transcribed as a result of functioning of the promoters or the gene products translated from those mRNAs.

When a structural gene is joined to the promoters of the invention downstream therefrom, the promoters can allow the structural gene to perform its function. The structural gene which can be used in the invention is not particularly restricted, but includes a gene encoding a enzyme involved in the synthesis of polyhydroxyalkanoate (particularly, copolymeric polyester P(3HB-co-3HH) resulting from copolymerization of 3-hydroxybutyric acid and 3-hydroxyhexanoic acid, a antibody gene, a gene encoding a useful protein such as lymphokine, or the like genes.

Furthermore, if necessary, the promoter of the invention and a structural gene joined downstream therefrom, together with a terminator, can be used as gene expression units. The terminator to be used here may be any appropriate one among the known terminators provided that it can be used in the objective expression system. Said terminator is not particularly restricted, but there may be mentioned ALK1 or GCN4, among others.

It is also possible to insert such gene expression units in a plasmid for utilization of the units. The plasmid which may be used in the present invention is not particularly restricted, but includes pUTU1 or pBTH10B (Nakazawa, et al., J. Bacteriol., vol. 79, 5030 (1997)), among others.

In accordance with the present invention, it is possible to transform the plasmid referred to above into host cells constituting an expression system for preparing transformed cells and then culture these transformed cells to cause expression of the structural gene transformed. In place of plasmid transformation, a DNA comprising any of the promoters of the invention and a structural gene joined downstream therefrom may be incorporated directly into the host chromosome. The host to be used here is not particularly restricted provided that at least one promoter of the invention can function therein, but a yeast belonging to the genus Candida is preferred, and Candida maltosa is particularly preferred.

The host cell can be transformed with the plasmid of the present invention in the conventional manner. For example, the calcium method (E. M. Lederberg, et al., J. Bacteriol., vol. 119, 1072 (1974)) or the electroporation method (Current Protocols in Molecular Biology, vol. 1, para. 1.8.4, 1994) can be used. Commercial transformation kits such as Fast Track™-Yeast Transformation Kit SM (Geno Technology) may also be utilized.

The function analysis of the novel promoters using the transformants obtained can be carried out by culturing them using a carbon source(s) utilizable by the same transformants. For the culture of said transformant, a medium generally used in the culture of a transformant may be used. Such medium may be prepared by adding a carbon source to YPD medium (enzyme extract 1%, polypepton 2%), YNB medium (Yeast nitrogen base 0.67%) or the like. In addition, a synthesized medium may be also used. The carbon source which may be used in the invention is not particularly restricted, but includes sugars such as glucose or maltose; n-alkanes, fatty acids, and oils and fats, among others. The temperature at the culture and culturing time are not particularly restricted as long as the condition is suitable for a transformant to growth. Preferably, said culture may be carried out at 10 to 40° C. for 2 through 72 hours. The function analysis of promoter of the cultured cell can be carried out by analyzing culture medium or proteins in the cell. There is no need to fracture the cell when an enzyme, which is expressed from a gene joined downstream from a promoter, or the like are expressed outside the cell. On the other hand, when the enzyme stays in the cell, it is necessary to treat it. The method for treating a cell is not particularly restricted. But, among them, there may be mentioned a method in which the cell is subjected to digestion by cell wall lytic enzyme, such as Zymolyase, and then to sonication, freeze-thawing, or the like treatment. Furthermore, direct physical fracturing with glass bead may be also used.

Function analysis of the promoter may be carried out by the method which can detect specifically an activity or a content of, such as an enzyme, which is expressed from a gene joined downstream from a promoter. There is no limitation for the analysis method, but the analysis can be carried out, for example, by constructing expression vectors for the expression of the Aeromonas caviae-derived enzyme (hereinafter referred to as “ORF2S”) involved in the synthesis of a copolymer of 3-hydroxybutyric acid and 3-hydroxyhexanoic acid, namely copolyester P (3HB-co-3HH), using the respective promoter regions cloned as described above under (1), and then determining the activity of that enzyme by the method described in the literature (Valentin, H. E., et al., Appl. Microbiol. Biotechnol., vol. 40, 699 (1994)). Particularly, the enzyme activity may be measured by a simple and easy manner in the case of a measurement of a promoter using ORF2S, by subjecting the COA-SH which becomes free on incorporation of (R)-3-hydroxybutylyl-CoA by ORF2S, to reaction with DTNB (5,5′-dithiobis(2-nitrobenzoic acid)), which can be reacted with COA-SH in equimolar ratio, and, at the same time, measuring increase per unit time of absorption at about 412 nm derived from ionized TNB (2-nitro-5-mercaptobenzoic acid) generated by the reaction with free CoA-SH.

By normalization using protein content in the fractured cell-containing solution, the value of enzyme activity obtained can be indicated as an enzyme specific activity expressed by the promoter of the invention; thus, it will be possible to compare a promoter activity of one another.

Among the promoters of the present invention, the ACT1 gene promoter, PMA1 gene promoter and TEF1 gene promoter are all promoters for the genes essential for growth and, therefore, can function under conditions under which Candida maltosa can grow, without being restricted by the carbon source species. The GAP3 gene promoter is the promoter for an enzyme involved in the glycolytic pathway and, from the viewpoint that very strong expression can be expected even when glucose is used as the carbon source, it has a property that any known promoter cannot have.

According to the production method of the present invention, said copolymeric polyester may be produced by culturing said transformed cell, the structural gene of which is the gene derived from Aeromonas caviae, encoding a enzyme involved in the synthesis of copolymeric polyester resulting from copolymerization of 3-hydroxybutyric acid and 3-hydroxyhexanoic acid.

As a carbon source which can be used in said culture, any one that can be utilized by the transformant may be used. In addition, culture medium containing other nutrition sources than carbon, such as nitrogen source, inorganic salts, or other culture medium containing an organic nutrition source, may be also used. Any temperature condition, in which the cell can grow, may be applied, but preferably it may be between 20 and 40° C. Culturing time is not particularly restricted, but it may be 1 to 7 days. Then, polyester may be recovered from the cultured cell or medium resulting from the culture.

As the carbon source, carbohydrate such as glucose, glycerin and sucrose, oils and fats, fatty acids, further n-paraffin and the like may be used. As the fats and oils, for instance, there maybe mentioned rapeseed oil, coconut oil, palm oil, palm kernel oil, among others. As the fatty acids, for instance, a saturated/unsaturated fatty acid such as hexanoic acid, octanoic acid, decanoic acid, lauric acid, oleic acid, palmitic acid, linoleic acid, linolenic acid, myristic acid, or a fatty acid derivative such as an ester or a salt of these fatty acid, may be mentioned. As one example, culture of Candida maltosa maybe carried out with oils and fats as a carbon source. In the case of a transformant which cannot utilize, or cannot effectively utilize, lipase maybe added to the medium to improve the utilization efficiency. Furthermore, oils and fats utilizing-ability can be attached by transforming lipase gene.

As the nitrogen source, for instance, ammonia, ammonium salt such as ammonium chloride, ammonium sulfate and ammonium phosphate; peptone, meat extract, yeast extract and the like may be mentioned. As inorganic salts, for instance, Dipotassium Hydrogenphosphate, Potassium Dihydrogenphosphate, magnesium phosphate, magnesium sulfate, sodium chloride and the like are included.

As the other organic nutrition sources, for instance, amino acids (i.e. glycine, alanine, serine, threonine, proline, etc.), vitamins (i.e. vitamin B1, vitamin B12, biotin, nicotinic-acid amide, pantothenic acid, vitamin C, etc.), and the like may be mentioned.

In the present invention, recovery of polyester from the cell may be carried out by the following method. After completion of the culture, the cell is separated from culture medium by centrifugal separator and the like, then the cell is washed with distilled water, methanol or the like, and dried. Polyester is extracted from the cell with an organic solvent such as chloroform. Cell constituent is removed from the organic solvent solution containing the polyester by, for example, filtration. A poor solvent such as methanol and hexane is added to thus-obtained filtrate to precipitate polyester. Supernatant is removed by filtration or centrifugation, and thus-obtained, precipitated polyester is dried to recover polyester.

### BRIEF DESCRIPTION OF THE DRAWINGS

FIG. 1 represents a restriction map of a plasmid named pUAL-ORF2S, which is used for the plasmid construction according to Example 9.

FIG. 2 represents a restriction map of a plasmid named pSTV-ALK1-ORF2S, which is used for the plasmid construction according to Examples 9 to 13.

FIG. 3 represents a restriction map of a plasmid named pUTA1, which is used for the plasmid construction according to Examples 9 to 13.

FIG. 4 represents a restriction map of a plasmid named pUTA-ALK1-ORF2S constructed in Example 9.

FIG. 5 represents a restriction map of a plasmid named pUTA-ACT1-ORF2S, which is one aspect of the invention, constructed in Example 10.

FIG. 6 represents a restriction map of a plasmid named pUTA-GAP3-ORF2S, which is one aspect of the invention, constructed in Example 11.

FIG. 7 represents a restriction map of a plasmid named pUTA-PMA1-ORF2S, which is one aspect of the invention, constructed in Example 12.

FIG. 8 is a schematic representation of a plasmid named pUTA-TEF1-ORF2S, which is one aspect of the invention, constructed in Example 13.

### BEST MODE FOR CARRING OUT THE INVENTION

The following examples illustrate the present invention more specifically. However, these examples are by no means limitative of the scope of the invention.

### EXAMPLE 1

### Preparation of Yeast Chromosomal DNAs

The chromosomal DNAs of Saccharomyces cerevisiae and Candida maltosa were prepared using E.Z.N.A. Yeast DNA Kits (products of OMEGA BIOTEK). The preparation procedure was as described in the protocol attached to the kits.

### EXAMPLE 2

### Amplification of Fragments of the ACT1, GAP3, PMA1 and TEF1 Genes of Saccharomyces cerevisiae

Fragments of the Saccharomyces cerevisiae ACT1, GAP3, PMA1 and TEF1 genes the base sequences of which are already known were amplified as follows. The fragments were amplified by PCR using synthetic DNAs specified under SEQ ID NOs: 1 and 2 (for ACT1), SEQ ID NOs: 3 and 4 (for GAP3), SEQ ID NOs: 5 and 6 (for PMA1) and SEQ ID NOs: 7 and 8 (for TEF1) as primers and the Saccharomyces cerevisiae chromosomal DNA prepared in Example 1 as a template together with TaKaRa Ex Taq polymerase (product of Takara Shuzo). The conditions employed were as described in the protocol attached to the kit. More specifically, water was added, to make 0.1 ml, to a mixture of 1 μg of the template DNA, two primers each in an amount to give a final concentration of 1 μM, 2.5 U of Ex Taq polymerase, 0.01 ml of the attached buffer and 0.008 ml of the attached dNTP mixture. This was subjected to 25 cycles of PCR (each cycle comprising: 15 seconds at 98° C., 1 minute at 55° C., and 1 minute at 72° C.) to thereby amplify an ACT1 gene fragment of about 400 bp, or a GAP3 gene fragment of about 1 kb, or a PMA1 gene fragment of about 2.8 kb, or a TEF1 gene fragment of about 1.5 kb, of Saccharomyces cerevisiae.

### EXAMPLE 3

### Preparation of Labeled Probe Fragments, Hybridization, Washing, and Detection of Positive Clones

The respective probe fragments amplified in the above manner were labeled with alkaline phosphatase using Amersham-Pharmacia' s Gene images AlkPhos kit according to the protocol attached thereto.

The hybridization was carried out overnight at 55° C. using Amersham-Pharmacia's Gene images AlkPhos kit according to the protocol attached thereto.

The washing was carried out at 55° C. and at room temperature using Amersham-Pharmacia' s Gene images AlkPhos kit according to the protocol attached thereto.

Positive clones were detected using Amersham-Pharmacia's CDP-Star kit according to the protocol attached thereto.

### EXAMPLE 4

### Cloning of the Candida Maltosa ACT1 Gene Promoter Region

The chromosomal DNA of Candida maltosa as prepared in Example 1 was cleaved with the restriction enzyme BglII and fragments of about 1 kb to 3 kb were extracted from the gel by following agarose gel electrophoresis. These fragments were joined to pUC19, which was treated with the restriction enzyme BamHI, and the resulting recombined plasmids were used to transform the Escherichia coli DH5α strain. About 3,000 transformed colonies were screened to hybridization with the alkaline phosphatase-labeled Saccharomyces cerevisiae ACT1 gene fragment as a probe. As a result, 6 colonies of positive clone were obtained and, upon partial base sequence determination of the inserted fragment, the insert was found to be a gene containing the ACT1 promoter region of Candida maltosa. The partial sequence of the cloned fragment is shown under SEQ ID NO: 9.

### EXAMPLE 5

### Cloning of the Candida Maltosa GAP3 Gene Promoter Region

The chromosomal DNA of Candida maltosa as prepared in Example 1 was cleaved with the restriction enzyme EcoRI and fragments of about 7 kb to 9 kb were extracted from the gel by following agarose gel electrophoresis. These fragments were joined to pUC19, which was treated with the same restriction enzyme, and the resulting recombined plasmids were used to transform the E. coli DH5α strain. About 2,000 transformed colonies were screened to hybridization with the alkaline phosphatase-labeled Saccharomyces cerevisiae GAP3 gene fragment as a probe. As a result, 2 colonies of positive clone were obtained and, upon partial base sequence determination of the inserted fragment, the insert was found to be a gene containing the GAP3 promoter region of Candida maltosa. The partial sequence of the cloned fragment is shown under SEQ ID NO: 10.

### EXAMPLE 6

### Cloning of the Candida Maltosa PMA1 Gene Promoter Region

The chromosomal DNA of Candida maltosa as prepared in Example 1 was cleaved with the restriction enzyme XbaI and fragments of about 2 kb to 4 kb were extracted from the gel by following agarose gel electrophoresis. These fragments were joined to pUC19, which was treated with the same restriction enzyme, and the resulting recombined plasmids were used to transform the E. coli strain DH5α. About 5,000 transformed colonies were screened to hybridization with the alkaline phosphatase-labeled Saccharomyces cerevisiae PMA1 gene fragment as a probe. As a result, 5 colonies of positive clone were obtained and, upon base sequence determination of the inserted fragment, the insert was found to be a gene containing the PMA1 promoter region of Candida maltosa, as shown under SEQ ID NO: 11.

### EXAMPLE 7

### Cloning of the Candida Maltosa TEF1 Gene Promoter Region

The chromosomal DNA of Candida maltosa as prepared in Example 1 was cleaved with the restriction enzymes EcoRI and PstI and fragments of about 2 kb to 4 kb were extracted from the gel by following agarose gel electrophoresis. These fragments were joined to pUC19 treated with the same restriction enzymes, and the resulting recombined plasmids were used to transform the E. coli DH5α strain. About 400 transformed colonies were screened to hybridization with the alkaline phosphatase-labeled Saccharomyces cerevisiae TEF1 gene fragment as a probe. As a result, 7 colonies of positive clone were obtained and, upon base sequence determination of the inserted fragment, the insert was found to be a gene containing the TEF1 promoter region of Candida maltosa, as shown under SEQ ID NO: 12.

### EXAMPLE 8

### Synthesis of a Gene Involved in Polyester Synthesis

An enzyme gene involved in polyester synthesis was synthesized based on the amino acid sequence of the Aeromonas caviae-derived polyhydroxyalkanoate (PHA) synthetase (T. Fukui, et al., FEMS Microbiology Letters, vol. 170, 69 (1999)). Since Candida maltosa is an yeast, which translates the CTG codon into serine, not into leucine, CTG was not assigned to the leucine codon. Those codons, which are frequently used in Candida maltosa, were preferentially selected as the codons corresponding to the respective amino acids. As for the frequency of the codon, the monograph “Nonconventional Yeasts in Biotechnology” written by Klaus Wolf (published by Springer) was consulted. The PHA-synthesizing enzyme gene (hereinafter referred to as “ORF2S” for short; SEQ ID NO: 13) was thus designed and the ORF2S gene portion was totally synthesized.

### EXAMPLE 9

### Construction of an ORF2S Expression Vector Using the Candida Maltosa ALK1 Promoter

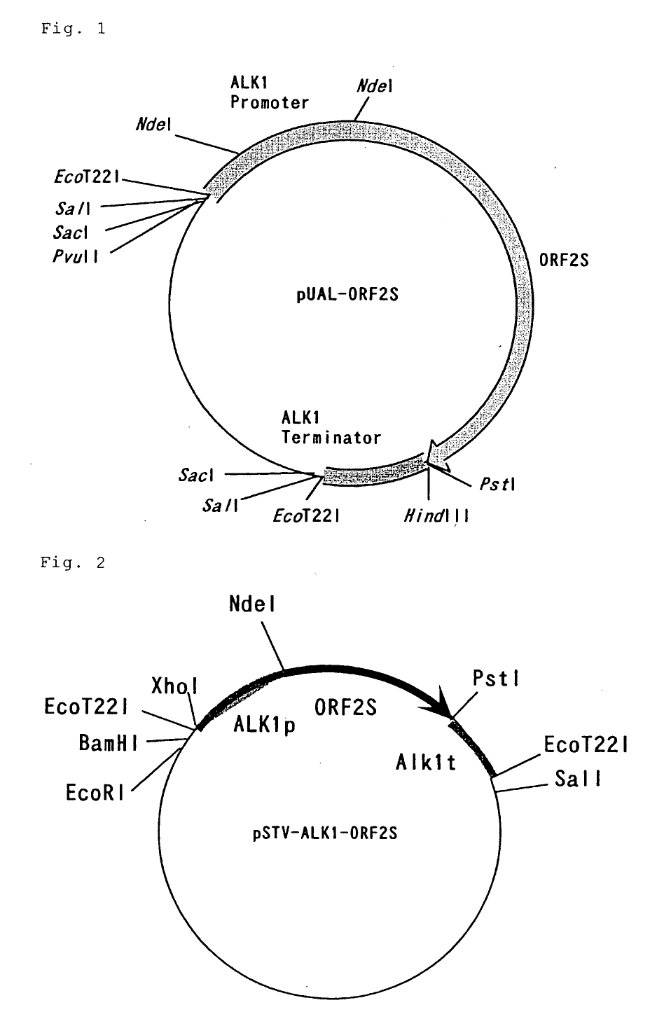

For the expression of the above ORF2S in Candida maltosa, the promoter ALK1p (SEQ ID NO: 15) of the Candida maltosa Alk1 gene was joined to the 5′ upstream thereof, and the terminator ALK1t (SEQ ID NO: 14) of the Candida maltosa Alk1 gene to the 3′ downstream thereof. The restriction enzyme sites for joining the promoter and terminator to the structural gene were produced by utilizing the PCR method. For the promoter portion, PCR was carried out using the DNAs shown under SEQ ID NO: 16 and SEQ ID NO: 17 with the promoter specified under SEQ ID NO: 15 as the template, and an ALK1p fragment having a PvuII site at the 5′ terminus and an EcoRI site at the 3′ terminus was prepared. For the terminator portion, PCR was carried out using the DNAs shown under SEQ ID NO: 18 and SEQ ID NO: 19 with the terminator specified under SEQ ID NO: 14 as the template, and an ALK1t fragment having a HindIII site at the 5′ terminus and an EcoRV site at the 3′ terminus was prepared. The ALK1p fragment was joined to pUCNT (described in WO 94/03613) at the PvuII-EcoRI site thereof, and the ALK1t fragment was joined to pUCNT at the HindIII-SspI site thereof, whereby pUAL1 was constructed. Then, the ORF2S fragment was joined to pUAL1 at the NdeI-PstI site thereof, whereby the plasmid pUAL-ORF2S (FIG. 1) was constructed. Then, this plasmid was partially cleaved with SalI, and the SalI site was converted to an XhoI site using an XhoI linker (product of Takara Shuzo) . The thus-modified plasmid was once cleaved with PvuI and PvuII and joined to the PvuI and SmaI fragment of pSTV28 (product of Takara Shuzo), whereby pSTV-ALK1ORF2S shown in FIG. 2 was constructed.

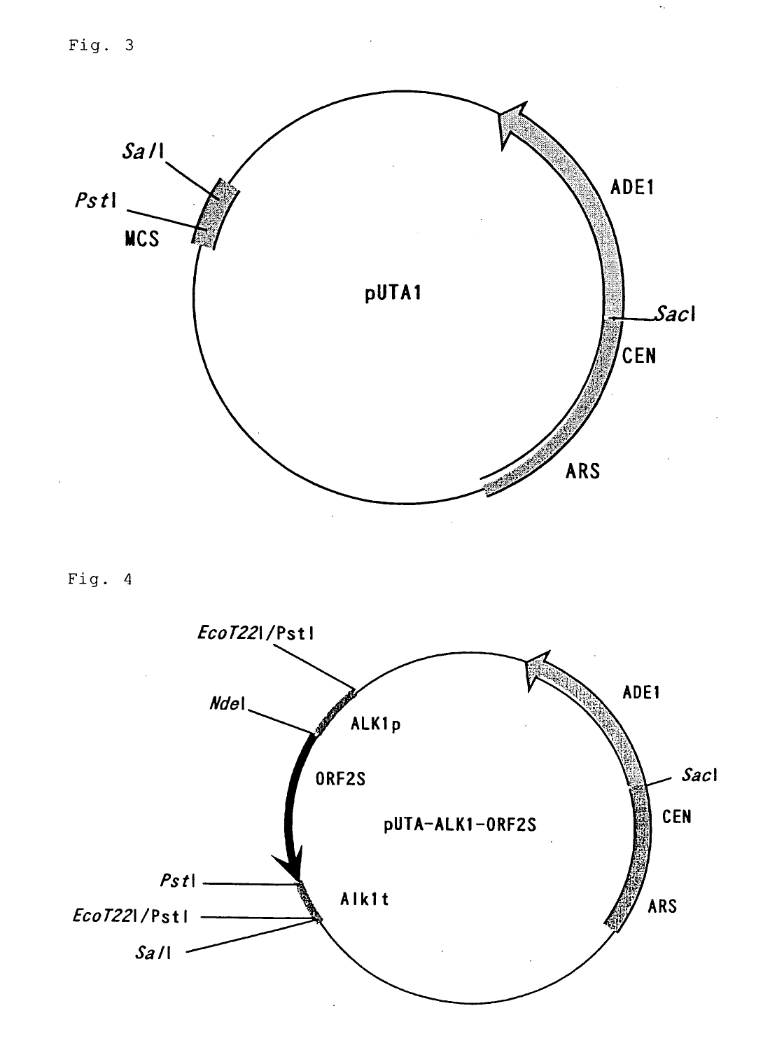

Furthermore, pUTA1 (FIG. 3), a vector, the marker gene of which was changed from uracil to adenine by using pUTU1 (M. Ohkuma, et al., J. Biol. Chem., vol. 273, 3948 (1998)) and the Candida maltosa ADE1 gene (Genebank D00855), was used. It was constructed by eliminating the URA3 gene from pUTU1 using XhoI and joining the ADE1 gene excised using SalI to the remainder.

A fragment containing the promoter, ORF2S and terminator was prepared from pSTV-ALK1-ORF2S using EcoT22I and inserted into pUTA1 at the PstI site thereof, whereby pUTA-ALK1-ORF2S shown in FIG. 4 was constructed.

### EXAMPLE 10

### Construction of an ORF2S Expression Vector Using the Candida Maltosa ACT1 Promoter

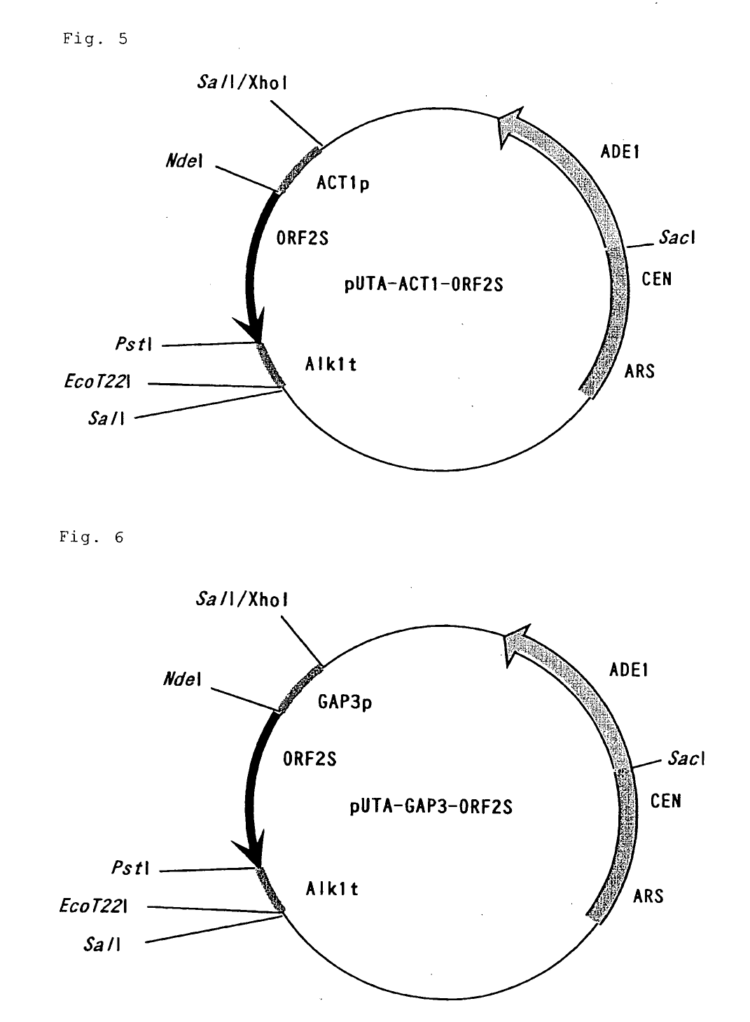

An ORF2S expression vector was constructed using the Candida maltosa ACT1 promoter region cloned in Example 4, as follows. A fragment having a restriction enzyme EcoT22I site at the 5′-side terminus and a restriction enzyme NdeI site at the 3′-side terminus was obtained by carrying out PCR using the synthetic DNAs shown under SEQ ID NOs: 20 and 21 as the primers and the Candida maltosa ACT1 promoter region-containing fragment (SEQ ID NO: 9) as the template, and this fragment was treated with EcoT22I and NdeI. pSTV-ALK1-ORF2S was treated in the same manner with EcoT22I and NdeI to prepare a fragment free of the ALK promoter. The two fragments were joined together to construct pSTV-ACT1-ORF2S. By treating this plasmid with the restriction enzyme EcoT22I, ACT1-ORF2S fragment was prepared. This fragment was inserted into the PstI site of the pUTA1 obtained in Example 9, whereby an expression vector, pUTA-ACT1-ORF2S, was constructed as shown in FIG. 5.

### EXAMPLE 11

### Construction of an ORF2S Expression Vector Using the Candida Maltosa GAP3 Promoter

An ORF2S expression vector was constructed using the Candida maltosa GAP3 promoter region cloned in Example 5, as follows. A fragment having a restriction enzyme XhoI site at the 5′-side terminus and a restriction enzyme NdeI site at the 3′-side terminus was obtained by carrying out PCR using the synthetic DNAs shown under SEQ ID NOs: 22 and 23 as the primers and the Candida maltosa GAP3 promoter region-containing fragment (SEQ ID NO: 10) as the template, and this fragment was treated with XhoI and NdeI. The pSTV-ALK1-ORF2S plasmid was treated in the same manner with XhoI and NdeI to prepare a fragment free of the ALK promoter. The two fragments obtained were joined together to construct pSTV-GAP3-ORF2S. This plasmid was treated with the restriction enzymes XhoI and SalI to prepare a GAP3-ORF2S fragment. The pUTA1 plasmid was treated with SalI. The two fragments thus obtained were joined together, whereby an expression vector, pUTA-GAP3-ORF2S, was constructed as shown in FIG. 6.

### EXAMPLE 12

### Construction of an ORF2S Expression Vector Using the Candida Maltosa PMA1 Promoter

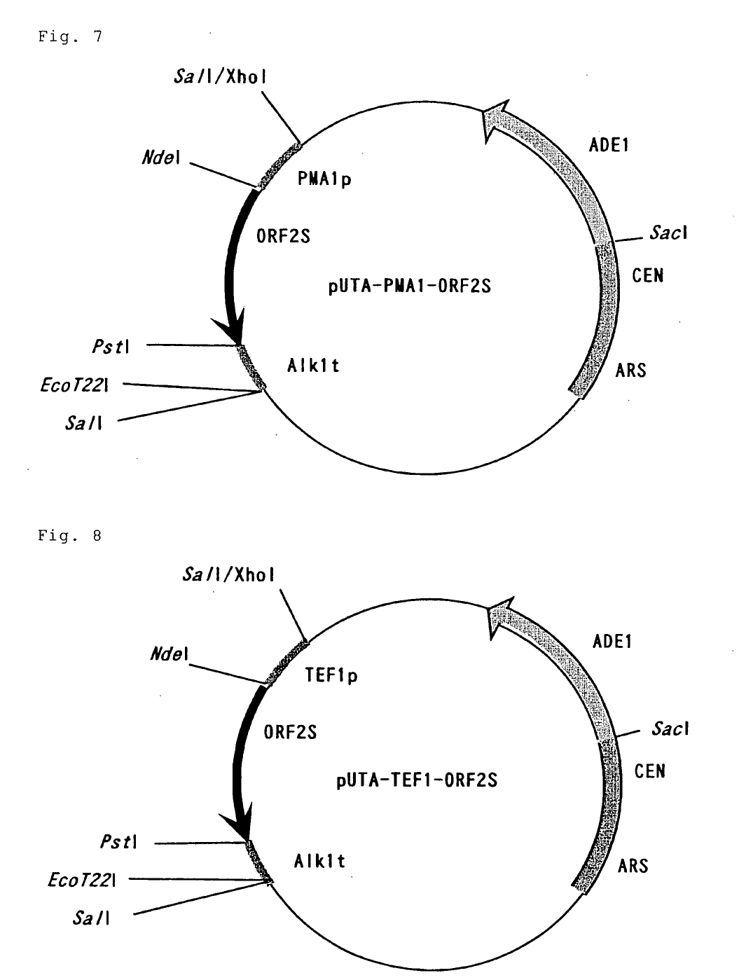

An ORF2S expression vector was constructed using the Candida maltosa PMA1 promoter region cloned in Example 6, as follows. A fragment having a restriction enzyme XhoI site at the 5′-side terminus and a restriction enzyme NdeI site at the 3′-side terminus was obtained by carrying out PCR using the synthetic DNAs shown under SEQ ID NOs: 24 and 25 as primers and the Candida maltosa PMA1 promoter region-containing fragment (SEQ ID NO: 11) as the template, and this fragment was treated with XhoI and NdeI. The pSTV-ALK1-ORF2S plasmid was treated in the same manner with XhoI and NdeI to prepare a fragment free of the ALK promoter. The two fragments obtained were joined together to construct pSTV-PMA1-ORF2S. This plasmid was treated with the restriction enzymes XhoI and SalI to prepare a PMA1-ORF2S fragment. The pUTA1 plasmid obtained in Example 9 was treated with SalI. Both the fragments obtained were joined together, whereby an expression vector, pUTA-PMA1-ORF2S, was constructed as shown in FIG. 7.

### EXAMPLE 13

### Construction of an ORF2S Expression Vector Using the Candida Maltosa TEF1 Promoter

An ORF2S expression vector was constructed using the Candida maltosa TEF1 promoter region cloned in Example 7, as follows. A fragment having a restriction enzyme XhoI site at the 5′-side terminus and a restriction enzyme NdeI site at the 3′-side terminus was obtained by carrying out PCR using the synthetic DNAs shown under SEQ ID NOs: 26 and 27 as the primers and the Candida maltosa TEF1 promoter region-containing fragment (SEQ ID NO: 12) as the template, and this fragment was treated with XhoI and NdeI. The pSTV-ALK1-ORF2S plasmid obtained in Example 9 was treated in the same manner with XhoI and NdeI to prepare a fragment free of the ALK promoter. The two fragments obtained were joined together to construct pSTV-TEF1-ORF2S. This plasmid was treated with the restriction enzymes XhoI and SalI to prepare a TEF1-ORF2S fragment. The pUTA1 plasmid obtained in Example 9 was treated with SalI. The two fragments thus obtained were joined together, whereby an expression vector, pUTA-TEF1-ORF2S, was constructed as shown in FIG. 8.

### EXAMPLE 14

### Isolation of a Candida Maltosa Transformed Colonies

The Candida maltosa AC16 strain (Deposit based on Budapest Treaty, International depository authority: National Institute of Advanced Industrial Science and Technology, International Patent Organism Depositary (1-1-3 Higashi, Tsukuba, Ibaraki, Japan), deposited on Nov. 15, 2000, accession No. FERM BP-7366) was transformed by the electroporation method. The organism was precultured overnight on YPD medium at 30° C. One milliliter of the culture fluid was inoculated into 100 ml of the same medium placed in a 500-ml Sakaguchi flask and cultured at 30° C. for about 7 hours. After 10 minutes of centrifugation at 3,000 rpm and at room temperature, cells were washed with three portions (each about 50 ml) of an ice-cooled 1 M sorbitol solution. The cells were suspended in 3 ml of the same solution, and the suspension was divided into 0.1-ml portions and stored at −80° C. until use as cells to be transformed. For transformation, ECM 600 M (product of BTX) was used. Specifically, 0.1 ml of the cells to be transformed and about 1 μg of any one of the expression vector DNAs constructed in Examples 9 to 13 were placed in a cuvette having a gap width of 2 mm, electric pulses were applied thereto under the condition: mode 2.5 kV, voltage 1.9 kV, and resistance 246Ω. Immediately thereafter, the cuvette was ice-cooled, 0.5 ml of 1 M sorbitol was added, and the mixture was maintained at room temperature for 1 hour and then cultured on a YNB selection plate at 30° C. The selection plate used was composed of 0.67 w/v % Yeast Nitrogen Base without amino acid (product of Difco), 2 w/v % glucose, and 2 w/v % Bacto Agar (product of Difco).

By the above operations, 5 kinds of transformants, each containing any one of the expression vector constructed in Example 9 to 13, were obtained.

### EXAMPLE 15

### Promoter Function Analysis

Each transformant obtained in Example 14 was precultured overnight on 0.67 w/v % Yeast Nitrogen Base without amino acid (product of Difco) plus 2 w/v % glucose. Then, 2.5 ml of the preculture was inoculated into 50 ml of the same medium placed in a 500-ml Sakaguchi flask and cultured at 30° C. for 24 hours. Only in a comparative example, namely in a culture of an ALK1 promoter-containing expression vector, 2 w/v % of n-dodecane was used as the carbon source. A portion, corresponding to about 10 ml, of the culture fluid was centrifuged at 3,000 rpm at room temperature for 10 minutes, and cells were washed with physiological saline and again centrifuged under the same conditions. The cell bodies were suspended in 1 ml of a 0.5 M potassium phosphate solution (pH 7.2), the suspension was mixed with the same volume of glass beads for fracturing yeast cells (0.45 mm; product of Biospec Products), and the cells were fractured by five times (each for 1 minute) of treatment on Mini Bead Beater (product of Biospec Products), followed by 10 seconds of centrifugation at 3,000 g on a centrifuge. The supernatant was recovered and used as an ORF2S activity assay sample. With the sample, the protein concentration was measured using Protein Assay Kit (product of BioRad) and the enzyme activity was determined as described in Valentin, H. E., et al., Appl. Microbiol. Biotechnol., vol. 40, 699 (1994).

Specifically, 0.01213 ml of aqueous solution containing 0.01 ml of cell fractured fluid and 7 mg/ml of (R)-3-hydroxybutylyl-CoA (product of Sigma), and 3.8 mg/ml of DTNB (product of Sigma) dissolved in 0.5 M potassium phosphate solution (pH 7.2) were added into 0.338 ml of water, and mixed. Then, absorbance variance at 412 nm of thus-obtained mixture was measured at room temperature, for 5 minutes. The measurement of the absorbance was carried out using a spectrophotometer produced by Shimadzu. The activity was calculated by the following formula.

Activity (U/ml)=ΔA₄₁₂/min×10³×V_{T}/ε₄₁₂×V_{E}

In the formula, ΔA₄₁₂/min represents an increase of absorbance per minute, V_{T} represents an amount of reaction mixture, ε₄₁₂ is 15.6×10³ (m⁻¹·cm⁻¹), and V_{E} represents an amount of an enzyme fluid.

The enzyme activity is expressed in terms of specific activity (U/mg) as in the above-cited document and the following formula.

Specific activity (U/mg)

=Activity(U/ml)/Protein Concentration (mg/ml)

The results thus obtained are shown in Table 1. From these results, it was revealed that the ACT1, GAP3, PMA1 and TEF1 promoter regions cloned in accordance with the present invention are promoters capable of functioning in Candida maltosa cells and are at least comparable in efficiency of the function to the ALK promoter.

TABLE 1

| Promoter | Enzyme activity (U/mg) |
|---|---|
| ACT1 | 0.021 |
| GAP3 | 0.018 |
| PMA1 | 0.020 |
| TEF1 | 0.025 |
| ALK1 | 0.021 |

### INDUSTRIAL APPLICABILITY

Thus, the present invention has made it possible to carry out highly efficient expression of useful genes in yeasts of the genus Candida, in particular in Candida maltosa.

1. An ACT1 gene promoter
which comprises a DNA selected from among the following
(a) to (d):
(a) a DNA shown under SEQ ID NO: 9;
(b) a DNA containing the base sequence shown under SEQ ID NO: 9 and having promoter activity;
(c) a DNA containing a base sequence derived from the base sequence shown under SEQ ID NO: 9 by deletion, substitution or addition of at least one base and having promoter activity;
(d) a DNA derived from a yeast belonging to genus Candida, which hybridizes with base sequence of SEQ ID NO: 9 under stringent condition and has a promoter activity.

2. A DNA
which comprises the ACT1 gene promoter according to claim 1 and a structural gene joined to the promoter sequence downstream therefrom.

3. A gene expression unit
which comprises the DNA according to claim 2 and a terminator.

4. A plasmid
which contains the gene expression unit according to claim 3.

5. The plasmid according to claim 4,
which is pUTA-ACT1-ORF2S.

6. A GAP3 gene promoter
which comprises a DNA selected from among the following
(a) to (d):
(a) a DNA shown under SEQ ID NO: 10;
(b) a DNA containing the base sequence shown under SEQ ID NO: 10 and having promoter activity;
(c) a DNA containing a base sequence derived from the base sequence shown under SEQ ID NO: 10 by deletion, substitution or addition of at least one base and having promoter activity.
(d) a DNA derived from a yeast belonging to genus Candida, which hybridizes with base sequence of SEQ ID NO: 10 under stringent condition and has a promoter activity.

7. A DNA
which comprises the GAP3 gene promoter according to claim 6 and a structural gene joined to the promoter sequence downstream therefrom.

8. A gene expression unit
which comprises the DNA according to claim 7 and a terminator.

9. A plasmid
which contains the gene expression unit according to claim 8.

10. The plasmid according to claim 9,
which is pUTA-GAP3-ORF2S.

11. A PMA1 gene promoter
which comprises a DNA selected from among the following
(a) to (d):
(a) a DNA shown under SEQ ID NO: 11;
(b) a DNA containing the base sequence shown under SEQ ID NO: 11 and having promoter activity;
(c) a DNA containing a base sequence derived from the base sequence shown under SEQ ID NO: 11 by deletion, substitution or addition of at least one base and having promoter activity.
(d) a DNA derived from a yeast belonging to genus Candida, which hybridizes with base sequence of SEQ ID NO: 11 under stringent condition and has a promoter activity.

12. A DNA
which comprises the PMA1 gene promoter according to claim 11 and a structural gene joined to the promoter sequence downstream therefrom.

13. A gene expression unit
which comprises the DNA according to claim 12 and a terminator.

14. A plasmid
which contains the gene expression unit according to claim 13.

15. The plasmid according to claim 14,
which is pUTA-PMA1-ORF2S.

16. A TEF1 gene promoter
which comprises a DNA selected from among the following
(a) to (d):
(a) a DNA shown under SEQ ID NO: 12;
(b) a DNA containing the base sequence shown under SEQ ID NO: 12 and having promoter activity;
(c) a DNA containing a base sequence derived from the base sequence shown under SEQ ID NO: 12 by deletion, substitution or addition of at least one base and having promoter activity.
(d) a DNA derived from a yeast belonging to genus Candida, which hybridizes with base sequence of SEQ ID NO: 12 under stringent condition and has a promoter activity.

17. A DNA
which comprises the TEF1 gene promoter according to claim 16 and a structural gene joined to the promoter sequence downstream therefrom.

18. A gene expression unit
which comprises the DNA according to claim 17 and a terminator.

19. A plasmid
which contains the gene expression unit according to claim 18.

20. The plasmid according to claim 19,
which is pUTA-TEF1-ORF2S.

21. A transformed cell as resulting from transformation of the DNA according to claim 2, 7, 12 or 17.

22. A transformed cell as resulting from transformation of the plasmid according to claim 4, 5, 9, 10, 14, 15, 19 or 20 into a host cell.

23. The transformed cell according to claim 21 or 22,
wherein the host cell is Candida maltosa.

24. The transformed cell according to any of claims 21 to 23,
wherein the structural gene is an Aeromonas caviae-derived gene encoding a enzyme involved in the synthesis of the copolymeric polyester resulting from copolymerization of 3-hydroxybutyric acid and 3-hydroxyhexanoic acid.

25. A method of producing the copolymeric polyester resulting from copolymerization of 3-hydroxybutyric acid and 3-hydroxyhexanoic acid which comprises culturing the transformed cell according to claim 24.

1. An ACT1 gene promoter which comprises a DNA selected from among the following (a) to (d):
(a) a DNA shown under SEQ ID NO: 9;
(b) a DNA containing the base sequence shown under SEQ ID NO: 9 and having promoter activity;
(c) a DNA containing a base sequence derived from the base sequence shown under SEQ ID NO: 9 by deletion, substitution or addition of at least one base and having promoter activity;
(d) a DNA derived from a yeast belonging to genus Candida, which hybridizes with base sequence of SEQ ID NO: 9 under stringent condition and has a promoter activity.

2. A DNA which comprises the ACT1 gene promoter according to claim 1 and a structural gene joined to the promoter sequence downstream therefrom.

3 A gene expression unit which comprises the DNA according to claim 2 and a terminator.

4. A plasmid which contains the gene expression unit according to claim 3.

5. The plasmid according to claim 4, which is pUTA-ACT1-ORF2S.

6. A GAP3 gene promoter which comprises a DNA selected from among the following (a) to (d):
(a) a DNA shown under SEQ ID NO: 10;
(b) a DNA containing the base sequence shown under SEQ I D NO: 10 and having promoter activity;
(c) a DNA containing a base sequence derived from the base sequence shown under SEQ ID NO: 10 by deletion, substitution or addition of at least one base and having promoter activity.
(d) a DNA derived from a yeast belonging to genus Candida, which hybridizes with base sequence of SEQ ID' NO: 10 under stringent condition and has a promoter activity.

7. A DNA which comprises the GAP3 gene promoter according to claim 6 and a structural gene joined to the promoter sequence downstream therefrom.

8. A gene expression unit which comprises the DNA according to claim 7 and a terminator.

9. A plasmid which contains the gene expression unit according to claim 8.

10. The plasmid according to claim 9, which is pUTA-GAP3-ORF2S.

11. A PMA1 gene promoter which comprises a DNA selected from among the following (a) to (d):
(a) a DNA shown under SEQ ID NO: 11;
(b) a DNA containing the base sequence shown under SEQ ID NO: 11 and having promoter activity;
(c) a DNA containing a base sequence derived from the base sequence shown under SEQ ID NO: 11 by deletion, substitution or addition of at least one base and having promoter activity.
(d) a DNA derived from a yeast belonging to genus Candida, which hybridizes with base sequence of SEQ ID NO: 11 under stringent condition and has a promoter activity.

12. A DNA which comprises the PMA1 gene promoter according to claim 11 and a structural gene joined to the promoter sequence downstream therefrom.

13. A gene expression unit which comprises the DNA according to claim 12 and a terminator.

14. A plasmid which contains the gene expression unit according to claim 13.

15. The plasmid according to claim 14, which is pUTA-PMA1-ORF2S.

16. A TEF1 gene promoter which comprises a DNA selected from among the following (a) to (d):
(a) a DNA shown under SEQ ID NO: 12;
(b) a DNA containing the base sequence shown under SEQ ID NO: 12 and having promoter activity;
(c) a DNA containing a base sequence derived from the base sequence shown under SEQ ID NO: 12 by deletion, substitution or addition of at least one base and having promoter activity.
(d) a DNA derived from a yeast belonging to genus Candida, which hybridizes with base sequence of SEQ ID NO: 12 under stringent condition and has a promoter activity.

17. A DNA which comprises the TEF1 gene promoter according to claim 16 and a structural gene joined to the promoter sequence downstream therefrom.

18. A gene expression unit which comprises the DNA according to claim 17 and a terminator.

19. A plasmid which contains the gene expression unit according to claim 18.

20. The plasmid according to claim 19, which is pUTA-TEF1-ORF2S.

21. A transformed cell as resulting from transformation of the DNA according to claim 2.

22. A transformed cell as resulting from transformation of the plasmid according to claim 4 into a host cell.

23. The transformed cell according to claim 21, wherein the host cell is Candida maltosa.

24. The transformed cell according to claim 21, wherein the structural gene is an Aeromonas caviae-derived gene encoding a enzyme involved in the synthesis of the copolymeric polyester resulting from copolymerization of 3-hydroxybutyric acid and 3-hydroxyhexanoic acid.

25. A method of producing the copolymeric polyester resulting from copolymerization of 3-hydroxybutyric acid and 3-hydroxyhexanoic acid which comprises culturing the transformed cell according to claim 24.
